(19) United States
(12) Patent Application Publication
(10) Pub. No.: US 2004/0198734 A1
(43) Pub. Date: Oct. 7, 2004

(54) 2-(4-Aryl or heteroaryl-piperazin-1-ylmethyl)-1H-indole derivatives interacting with the dopamine

(75) Inventors: Anton Franz Josef Fliri, Stonington, CT (US); Mark Jerome Majchrzak, East Lyme, CT (US); Patricia Ann Seymour, Westerly, RI (US); Stevin Howard Zorn, Ann Arbor, MI (US); Hans Rollema, Nystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/825,406

(22) Filed: Apr. 15, 2004

Related U.S. Application Data
(63) Continuation of application No. 10/340,699, filed on Jan. 10, 2003, now abandoned.
Application No. 10/340,699 is a continuation of application No. 09/842,569, filed on Apr. 25, 2001, now abandoned.

(51) Int. Cl. A61K 31/53; C 07D 4 3/14; C07D 487/14

(52) U.S. Cl. 514/242; 514/243; 514/252.19; 544/182; 544/183; 544/295

(57) ABSTRACT

A compound of the formula

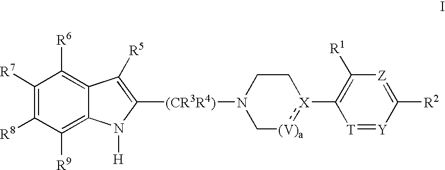

wherein a, T, V, X, Y, Z, R¹, R², R³, R⁴, R⁵, R⁶, R⁷, R⁸ and R⁹ are as defined above, their pharmaceutically acceptable salts and pharmaceutical compositions containing such compounds or their salts.

### BACKGROUND OF THE INVENTION

The present invention relates to 2-(4-aryl or heteroaryl-piperazin-1-ylmethyl)-1H-indole derivatives possessing central dopaminergic activity. Such compounds are useful in the treatment of Central Nervous Systems (CNS) disorders. This invention also relates to a method of using such compounds in the treatment of the above disorders in mammals, especially humans, and the pharmaceutical compositions useful therefor.

It is generally known that dopamine receptors seem to be important for many functions in the animal body. For example, altered functions of these receptors participate in the genesis of psychosis, drug addiction, compulsive disorders, bipolar disorders, vision, emesis, sleep, feeding, learning, memory, sexual behavior, regulation of immunological responses and blood pressure. Since these receptors control a great number of pharmacological events, not all of them are presently known, there is a possibility that compounds acting preferentially on D4 dopamine receptor may exert a wide range of therapeutic effects in humans.

The 2-(4-aryl or heteroaryl-piperazin-1-ylmethyl)-1H-indole derivatives of the present invention, including forms of tautomers, enantiomers and acceptable acid addition salts, are centrally acting D4-dopamine receptor agonists and thus are useful as cognition enhancers and treatment of CNS diseases, such as Parkinsons disease, Alzheimer's disease, learning and memory abnormalities. Another feature of this invention provides for the use of combinations of compounds of the present invention in conjunction with D1, D2, D3 or D5 dopamine receptor agonists, such as L-dopa and D2 agonists, in treatment of CNS diseases, such as Parkinson's disease, Alzheimer's disease, attention deficit disorder and learning and memory abnormalities.

### SUMMARY OF THE INVENTION

The present invention relates to a compound of the formula

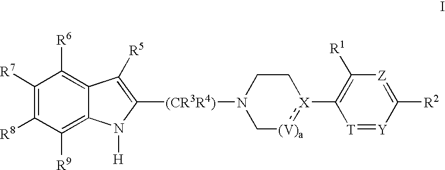

or the pharmaceutically acceptable salt thereof, wherein the broken line represents an optional double bond;

a is 0 or 1, wherein when a is 0, X may form an optional double bond with the carbon adjacent to V;

V is CHR¹⁰ wherein R¹⁰ is hydrogen or (C₁-C₆)alkyl;

T is nitrogen or CH;

X is nitrogen or CR¹¹ wherein R¹¹ is hydrogen, (C₁-C₆)alkyl, (C₁-C₆)alkoxy, hydroxy or cyano;

Y and Z are each independently nitrogen or CR¹² wherein R¹² is hydrogen, chloro, bromo, trifluoromethyl, trifluoromethoxy, cyano, (C₁-C₆)alkoxy or (C₁-C₆)alkyl;

R¹ is hydrogen, fluoro, chloro, bromo, trifluoromethyl, trifluoromethoxy, cyano or (C₁-C₆)alkyl;

R², R⁶, R⁷, R⁸ and R⁹ are each independently selected from hydrogen, fluoro, chloro, bromo, trifluoromethyl, trifluoromethoxy, cyano, (C₁-C₆)alkoxy and (C₁-C₆)alkyl;

R³ and R⁴ are each independently hydrogen or (C₁-C₆)alkyl; and

R⁵ is hydrogen, (C₁-C₆)alkoxy, trifluoromethyl, cyano, (C₁-C₆)alkyl or R¹³CO— wherein R¹³ is amino, (C₁-C₆)alkylamino, ((C₁-C₆)alkyl)₂amino, (C₁-C₆)alkyl, (C₈-C₁₀)aryl;

or when a is 1, R¹ and R¹⁰ may be taken together with the carbons to which they are attached to form a compound of the formula

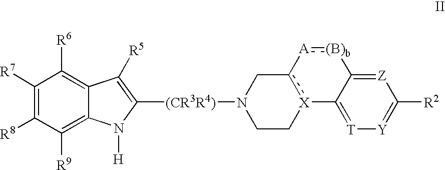

wherein the broken lines represent optional bonds;

T, X, Y, Z, R², R³, R⁴, R⁵, R⁶, R⁷, R⁸ and R⁹ are defined as above;

b is 0 or 1; and

A and B are each independently CH, CH₂, oxygen, sulfur, NH or nitrogen;

with the proviso that when X is nitrogen, the optional double bond between X and V does not exist;

with the proviso that when b is 0, the optional double bond between A and B does not exist; and

with the proviso that when b is 1, A and B cannot both be oxygen or sulfur.

The term “alkyl”, as used herein, unless otherwise indicated, includes saturated monovalent hydrocarbon radicals having straight, branched or cyclic moieties or combinations thereof.

The term “alkoxy”, as used herein, includes O-alkyl groups wherein “alkyl” is defined above.

The term “treating”, as used herein, refers to reversing, alleviating, inhibiting the progress of, or preventing the disorder or condition to which such term applies, or one or more symptoms of such disorders or condition. The term “treatment”, as used herein, refers to the act of treating, as “treating” is defined immediately above.

The term “disorders of the dopamine system”, as referred to herein, refers to disorders the treatment of which can be effected or facilitated by altering (i.e., increasing or decreasing) dopamine mediated neurotransmission.

The compounds in accordance with the present invention, being ligands for dopamine receptor subtypes, especially the dopamine D4 receptor, within the body, are accordingly of use in the treatment of disorders of the dopamine system.

The compound of formula I may have chiral centers and therefore exist in different enantiomeric forms. This invention relates to all optical isomers and stereoisomers of the compounds of formula I and mixtures thereof.

Preferred compounds of formula I include those wherein X is nitrogen.

Other preferred compounds of formula I include those wherein Y and Z are each CR¹² wherein R¹² is hydrogen or fluoro.

Other preferred compounds of formula I include those wherein R² is hydrogen, fluoro or chloro.

Other preferred compounds of formula I include those wherein R³, R⁴ and R⁵ are hydrogen.

Other preferred compounds of formula I include those wherein R⁷ is fluoro or chloro.

Other preferred compounds of formula I include those wherein R⁹ is fluoro, chloro, bromo or alkoxy.

More preferred compounds of formula I include those wherein X is nitrogen; Y and Z are each CR¹³ wherein R¹³ is hydrogen or fluoro; R² is hydrogen fluoro or chloro; R³, R⁴ and R⁵ are hydrogen; R⁷ is fluoro or chloro; and R⁹ is fluoro, chloro, bromo or alkoxy.

Specific preferred compounds of formula I include the following:

2-[4-(3-Trifluoromethyl-phenyl)-piperazin-1-ylmethyl]-1H-indole;

5-Fluoro-2-[4-(3-trifluoromethyl-phenyl)-piperazin-1-ylmethyl]-1H-indole;

5-Fluoro-2-[4-(4-fluoro-phenyl)-piperazin-1-ylmethyl]-1H-indole;

5-Fluoro-2-[4-(4-fluoro-phenyl)-piperazin-1-ylmethyl]-1H-indole;

5-Fluoro-2-(4-pyridin-2-yl-piperazin-1-ylmethyl)-1H-indole;

2-[4-(6-Chloro-pyridazin-3-yl)-piperazin-1-ylmethyl]-5-fluoro-1H-indole;

5-Fluoro-2-(4-[5′-fluoro]pyridin-2-yl-piperazin-1-ylmethyl)-1H-indole;

2-(4-pyridin-2-yl-piperazin-1-ylmethyl)-1H-azaindole;

5-Fluoro-2-(4-pyridin-2-yl-piperazin-1-ylmethyl)-1H-azaindole; and

2-[4-(4-fluoro-phenyl)-piperazin-1-ylmethyl]-1H-azaindole.

The present invention also relates to a method for treating disorders of the dopamine system including psychotic disorders (affective psychosis, schizophrenia, and schizoaffective disorders), movement disorders (extrapyramidal side effects from neuroleptic agents, neuroleptic malignant syndrome, tardive dyskinesia, Gilles De La Tourette's syndrome, Parkinson's disease or Huntington's disease), gastrointestinal disorders (gastric acid secretion or emesis), chemical abuse, chemical dependencies, substance abuse, vascular and cardiovascular disorders (congestive heart failure and hypertension), ocular disorders and sleep disorders in a mammal, comprising administering to said mammal an amount of a D4 dopamine receptor selective compound according to formula I, or a pharmaceutically acceptable salt thereof, that is effective in treating such disorder.

The present invention also relates to a method for treating disorders of the dopamine system including psychotic disorders (affective psychosis, schizophrenia, and schizoaffective disorders), movement disorders (extrapyramidal side effects from neuroleptic agents, neuroleptic malignant syndrome, tardive dyskinesia, Gilles De La Tourette's syndrome, Parkinson's disease or Huntington's disease), gastrointestinal disorders (gastric acid secretion or emesis), chemical abuse, chemical dependencies, substance abuse, vascular and cardiovascular disorders (congestive heart failure and hypertension), ocular disorders and sleep disorders in a mammal, comprising administering to said mammal an amount of a D4 dopamine receptor selective compound according to formula I, or a pharmaceutically acceptable salt thereof, in conjunction with one or more D1, D2, D3 or D5 dopamine receptor agonists, that is effective in treating such disorder.

The present invention also relates to a pharmaceutical composition for treating disorders of the dopamine system including psychotic disorders (affective psychosis, schizophrenia, and schizoaffective disorders), movement disorders (extrapyramidal side effects from neuroleptic agents, neuroleptic malignant syndrome, tardive dyskinesia, Gilles De La Tourette's syndrome, Parkinson's disease or Huntington's disease), gastrointestinal disorders (gastric acid secretion or emesis), chemical abuse, chemical dependencies, substance abuse, vascular and cardiovascular disorders (congestive heart failure and hypertension), ocular disorders and sleep disorders in a mammal, comprising administering to said mammal an amount of a D4 dopamine receptor selective compound according to formula I, or a pharmaceutically acceptable salt thereof, that is effective in treating such disorder.

The present invention also relates to a pharmaceutical composition for treating disorders of the dopamine system including psychotic disorders (affective psychosis, schizophrenia, and schizoaffective disorders), movement disorders (extrapyramidal side effects from neuroleptic agents, neuroleptic malignant syndrome, tardive dyskinesia, Gilles De La Tourette's syndrome, Parkinson's disease or Huntington's disease), gastrointestinal disorders (gastric acid secretion or emesis), chemical abuse, chemical dependencies, substance abuse, vascular and cardiovascular disorders (congestive heart failure and hypertension), ocular disorders and sleep disorders in a mammal, comprising administering to said mammal an amount of a D4 dopamine receptor selective compound according to formula I, or a pharmaceutically acceptable salt thereof, in conjunction with one or more D1, D2, D3 or D5 dopamine receptor agonists, that is effective in treating such disorder.

### DETAILED DESCRIPTION OF THE INVENTION

The following reaction Schemes illustrate the preparation of the compounds of the present invention. Unless otherwise indicated a, T, V, X, Y, Z, R¹, R¹, R², R³, R⁴, R⁵, R⁶, R⁷, R⁸ and R⁹ in the reaction Schemes and the discussion that follow are defined as above.

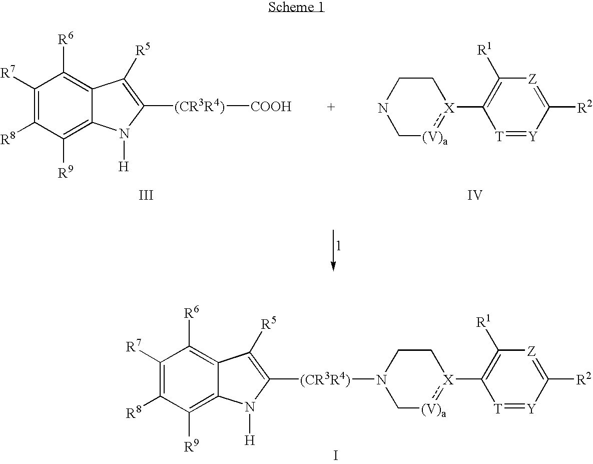

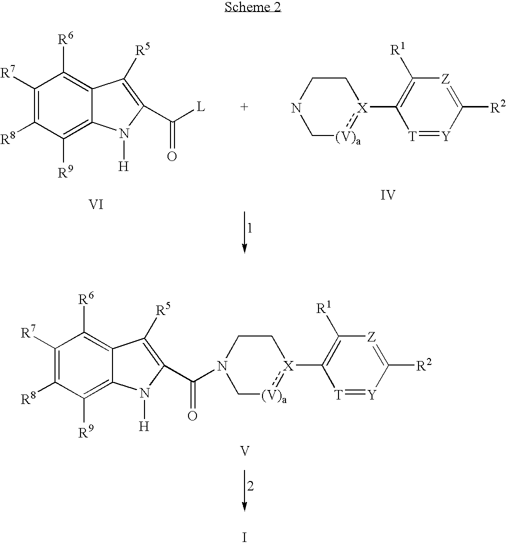

In reaction 1 of Scheme 1, the compounds of formula III and IV are coupled to form the corresponding compound of formula I by first treating III with O-, N-dimethyl hydroxylamine hydrochloride, dicyclohexylcarbodiimide and a base, such as triethylamine, in a polar aprotic solvent, such as methylene chloride. The hydroxamide intermediate so formed is reduced, using a reducing agent such as lithium aluminum hydride, in a polar aprotic solvent, such as tetrahydrofuran. The reductive amination of the aldehyde intermediate so formed is accomplished by reacting the aldehyde with the compound of the formula IV in the presence of sodium triacetoxyborohydride and a polar aprotic solvent, such as dichloroethane. The reaction mixture is stirred, under inert atmosphere, at room temperature for a time period between about 40 hours to about 56 hours, preferably about 48 hours.

In reaction 1 of Scheme 2, the compounds of formula VI, wherein L is a leaving group such as chloro, bromo, methoxy or any activated ester derivative such as para-nitro phenyl ester, hydroxy benzotriazole ester, N-hydroxysuccinimide ester or hydroxy, and IV are coupled to form the corresponding methanone compound of formula III by reacting VI and IV in the presence of diisopropylethylamine, carbodiimide or a dehydrating agent and a polar aprotic solvent, such as methylene chloride, or in form of mixtures containing, if desired, combinations of organic solvents or water such as combinations of cyclic and acyclic mono and dialkylamides, (C₁-C₄) alcohols, halogenated solvents, or acyclic and cyclic alkylethers at temperatures ranging from about 0° C. to about 150° C., preferabley about 0° C. or the boiling point of the same solvent mixture. Addition of an acid acceptor such as an alkalicarbonate, a tertiary amine or a similar reagent may be useful.

In reaction 2 of Scheme 2, the methanone compound of formula V is converted to the corresponding compound of formula I, wherein R and R⁴ are hydrogen, by reducing V with a reducing agent, such as lithium aluminum hydride or a borane derivative, in the presence of a polar aprotic solvent, such as tetrahydrofuran, for a time period between about 10 hours to about 14 hours, preferably about 12 hours.

In each of the above reactions, pressure is not critical. Pressures in th range of about 0.5 atmospheres to 3 atmospheres are suitable, and ambient pressure (generally, about one atmosphere) is preferred as a matter of convenience. Also, for those reactions where the preferred temperature varies with the particular compounds reacted, no preferred temperature is stated. For such reactions, preferred temperatures for particular reactants may be determined by monitoring the reaction using thin layer chromatography.

The novel compounds of the formula I and the pharmaceutically acceptable salts thereof (herein “the therapeutic compounds of this invention”) are useful as dopaminergic agents, i.e., they possess the ability to alter dopamine mediated neurotransmission in mammals, including humans. They are therefore able to function as therapeutic agents in the treatment of a variety of conditions in mammals, the treatment or prevention of which can be effected or facilitated by an increase or decrease in dopamine mediated neurotransmission.

The compounds of the formula I that are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

The therapeutic compounds of this invention can be administered orally, transdermally (e.g. through the use of a patch), parenterally or topically. Oral administration is preferred. In general, these compounds are most desirably administered in dosages ranging from about 0.1 mg up to about 1000 mg per day, or 1 mg to 1000 mg per day in some cases, although variations may occur depending on the weight and condition of the person being treated and the particular route of administration chosen. In some instances, dosage levels below the lower limit of the aforesaid range may be more than adequate, while in other cases still larger doses may be employed without causing any harmful side effect, provided that such larger doses are first divided into several small doses for administration throughout the day.

The therapeutic compounds of the invention may be administered alone or in combination with pharmaceutically acceptable carriers or diluents by either of the two routes previously indicated, and such administration may be carried out in single or multiple doses. More particularly, the novel therapeutic compounds of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Such carriers include solid diluents or fillers, sterile aqueous media and various non-toxic organic solvents, for example. Moreover, oral pharmaceutical compositions can be suitably sweetened and/or flavored.

For oral administration, tablets containing various excipients such as microcrystalline cellulose, sodium citrate, calcium carbonate, dicalcium phosphate and glycine may be employed along with various disintegrants such as starch (and preferably corm, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is also possible to administer the compounds of the present invention topically when treating inflammatory conditions of the skin and this may preferably be done by way of creams, jellies, gels, pastes, ointments and the like, in accordance with standard pharmaceutical practice.

The ability of compounds to bind to mammalian dopamine receptors, and the relative ability of compounds of this invention to inhibit [³H]-spiperone binding to human dopamine D₄ receptor subtypes expressed in clonal cell lines was measured using the following procedure.

D₄ Receptor Binding Ability

The determination of D₄ receptor binding ability has been described by Van Tol, et al. (Nature, 1991, 350, 610). Clonal cell lines expressing the human dopamine D₄ receptor are harvested and homogenized (polytron) in a 50 mM Tris:HCl (pH 7.4 at 4° C.) buffer containing 5 mM EDTA, 1.5 mM calcium chloride (CaCl₂), 5 mM magnesium chloride (MgCl₂), 5 mM potassium chloride (KCl) and 120 mM sodium chloride (NaCl). The homogenates are centrifugated for 10-15 min. at 48,000 g, and the resulting pellets resuspended in a buffer at a concentration of 150-250 mg/ml. For saturation experiments, 0.75 ml aliquots of tissue homogenate are incubated in triplicate with increasing concentrations of [³H]-spiperone (70.3 Ci/mmol; 10-3000 pM final concentration) for 30-120 minutes at 22° C. in a total volume of 1 ml. For competition binding experiments, assays are initiated by the addition of 0.75 ml of membrane and incubated in duplicate with the indicated concentrations of competing ligands (10⁻¹⁴-10⁻³ M) and/or [³H]-spiperone (100-300 pM) for 60-120 min at 22° C. Assays are terminated by rapid filtration through a Brandell cell harvester and the filters subsequently monitored for tritium as described by Sunahara, R. K. et al. (Nature, 1990, 346, 76). For all experiments, specific [³H]spiperone binding is defined as that inhibited by 1-10 mM (+)butaclamol. Binding data are analyzed by non-linear least square curve-fitting. The compounds of the Examples were tested in this assay, and all were found to have binding affinities (Kᵢ) for the displacement of [³H]-spiperone of less than 2 micromolar.

Human D4 Receptor Modulation of cAMP Formation

Chinese hamster ovary (CHO) cells expressing the human D4.4 dopamine receptor were obtained from Dr. H. Van Tol (Clarke Institute of Psychiatry, Toronto), and were grown to confluence in Minimal Essential Alpha Media (Gibco) supplemented with 2.5% Fetal Bovine Serum (not heat inactivated), 2.5% Equine Serum (heat inactivated), and 500 μg/ml Geneticin. Monolayers were disrupted and cells disloged with 5 mM ethylenediaminetetraacetic acid (EDTA) and resuspended in phosphate buffered saline buffer containing 5 mM magnesium chloride, 30 mM hydroxyethylpiperazine-N-ethanesulfonic acid (HEPES), 300 μM 3-isobutyl-1-methyl-xanthine (IBMX, a phosphodiesterase inhibitor), and 5.6 mM dextrose. Cells (approximately 200,000/tube) were exposed to 5 μM forskolin (an adenylate cyclase activator), forskolin plus test compounds or quinpirole (a D4 receptor agonist), or forskolin plus quinpirole plus antagonist for 11 minutes. In experiments with antagonists, cells were exposed to antagonists 11 minutes prior to agonist challenge. The effect of test compounds in the absence of the agonist quinpirole was used to judge agonist activity. D4 agonists produce an inhibition of cAMP accumulation which can be reversed by D4 receptor antagonists. The reaction was terminated with the addition of 6N perchloric acid, and samples neutralized with 5N potassium hydroxide and 2M Tris buffer. Cyclic AMP levels were measured using a commercially available competitive binding kit (Amersham). IC₅₀ values were calculated by linear regression analysis of the concentration-response curves. Kᵢ values were calculated using the equation: Kᵢ=IC₅O/(1+[agonist]/[agonist EC₅₀]} (Minneman and Johnson, 1984).

The present invention is illustrated by the following examples, but it is not limited to the details thereof.

### EXAMPLE 1

### 2-[4-(6-Chloro-pyridazin-3-yl)-piperazin-1-ylmethyl]-5-fluoro-1H-indole

A mixture of 5 gm of 5-fluoro 2 indole carboxylic acid, 2.74 gm of O-, N-dimethyl hydroxylamine hydrochloride, 3.89 ml triethylamine and 5.76 gm of dicyclohexylcarbodiimide in 35 ml methylene chloride is stirred at ambient temperature until a tan precipitate is formed. The solid is removed by filtration, the residue concentrated and purified on SiO2 (25%) EtOAc in Hexane) obtained are 3.6 gm (64%) of the N—O-dimethyl 2 indole hydroxamide. 3.9 gm of N—O-dimethyl 2 indole hydroxylamide is added over a period of 5 minutes to a cold suspension (−40 C) of 0.67 gm LiAIH4 in 30 ml tetrahydrofuran. The mixture is stirre for an hour (40 C->−30 C) treated with a saturated aqueous solution of sodium sulfate and warmed to ambient temperature. The solvent is separated after addition of solid sodiumsulfate and concentrated until a solid precipitate is formed (2.94 gm of 5-fluoro 2-indolecarboxaldehyde.

A mixture of 0.96 gm of 4-(5-chloro-phenyl)-piperazine, 1.0 gm of 5-Fluoro, 2-indolecarboxaldehyde and 1.2 gm of sodium triacetoxyborohydride in 50 ml dichloroethane is stirred under nitrogen at ambient temperature for 48 hours. The solvent is removed and the residue portioned between 100 ml EtOAc and 20 ml NaOH (1N). The organic layer is washed with water (2×20 ml) and brine (1×10 ml) and concentrated. The residue is purified on SiO2 (eluent: 5% methanol in methylene chloride) to yield 1.02 gm of a cream colored solid which has a mp.: 204-205 C°).

### EXAMPLE 2

### 5-Fluoro-1H-indol-2-yl)-[4-(3-trifluoromethyl-phenyl)-piperazin-1-yl]-methanone

A mixture of 1.0 mmol of 5-fluoro, 2-indole carboxylicacidchloride and 230 mg of meta-trifluoromethylphenylpiperazine and 129 mg of diisopropylethylamine in 10 ml methylenchloride is kept at ambient temperature for 12 hours. Water is added, the organic layers separated, washed with wate, dried over sodium sulfate and concentrated to yield 296 mg of the title compound. MP: 198° C.

### EXAMPLE 3

### 5-Fluoro-2-[4-(3-trifluoromethyl-phenyl)-piperazin-1-ylmethyl]-1H-indole hydrochloride

A solution of 275 mg of 5-Fluoro-1H-indol-2-yl)-[4-(3-trifluoromethyl-phenyl)-piperazin-1-yl]methanone in 5 ml anhydrous tetrahydrofurane is kept under an inert gas atmosphere and is treated at ambient temperature with 2.11 ml of a 1M solution of Lithiumaluminumhydride in tetrahydrofurane. After 12 hours the mixture is treated with 78 μl 15% Sodium Hydroxide solution and again 234 μl water. After addition of magnesiumsulfate the organic layer is separated and concentrated to a yellow oil (240 mg). This oil is disolved in ether and treated with an ether solution of hydrochloric acid until a precipitate is formed. The precipitate is collected, dried under vacuum.

The title compounds of Examples 4- were prepared by a methods analogous to that described in Example 1-3.

### EXAMPLE 4

### 2-[4-(3-Trifluoromethyl-phenyl)-piperazin-1-ylmethyl]-1H-indol-5-ol

MP: 188-190° C.; HRSMS 375.15.

### EXAMPLE 5

### 2-[4-(3-Trifluoromethyl-phenyl)-piperazin-1-ylmethyl]-1H-indole

MP: 192-194° C.; HRSMS 359.15.

### EXAMPLE 6

### (1H-Indol-2-yl)-[4-(2-nitro-phenyl)-piperazin-1-yl]-methanone

MP: 186-189° C.

### EXAMPLE 7

### (5-Fluoro-1H-indol-2-yl)-[4-(2-nitro-phenyl)-piperazin-1-yl]-methanone

MP: 184-188° C.

### EXAMPLE 8

### (5-Fluoro-1H-indol-2-yl)-[4-(3-trifluoromethyl-phenyl)-piperazin-1-yl]-methanone

MP: 198° C.

### EXAMPLE 9

### 3-[4-(1H-Indol-2-ylmethyl)-piperazin-1-yl]-benzo[d]isothiazole

MP: 150-152° C.; MRSMS 348.12.

### EXAMPLE 10

### 5-Fluoro-2-[4-(3-trifluoromethyl-phenyl)-piperazin-1-ylmethyl-1H-indole

MP: 196-197° C.; HRSMS 377.148.

### EXAMPLE 11

### 2-(4-Naphthalen-1-yl-piperazin-1-ylmethyl)-1H-indole

MP: 238-239° C.; HRSMS 341.19.

### EXAMPLE 12

### 2-[4-(2-Nitro-phenyl)-piperazin-1-ylmethyl]-1H-indole

MP: 210-211° C.; HRSMS 336.16.

### EXAMPLE 13

### 5-Fluoro-2-[4-(2-nitro-phenyl)-piperazin-1-ylmethyl]-1H-indole

MP: 236° C.; HRSMS 354.14.

### EXAMPLE 14

### 5-Fluoro-2-(4-naphthalen-1-yl-piperazin-1-ylmethyl)-1H-indole

MP: 249-250° C.; HRSMS 359.18.

### EXAMPLE 15

### 5-Fluoro-2-(4-pyridin-2-yl-piperazin-1-ylmethyl)-1H-indole

MP: 242° C.; HRSMS 310.15.

### EXAMPLE 16

### 5-Fluoro-2-[4-(4-fluoro-phenyl)-piperazin-1-ylmethyl]-1H-indole

MP:

### EXAMPLE 17

### 5-Fluoro-2-(4-pyrimidin-2-yl-piperazin-1-ylmethyl)-1H-indole

MP: 199° C.; HRSMS 311.16.

### EXAMPLE 18

### (5-Fluoro-1H-indol-2-yl)-(4-pyridin-2-yl-piperazin-1-yl)-methanone

MP: 214-218° C.

### EXAMPLE 19

### 2-(4-Pyridin-2-yl-piperazin-1-ylmethyl)-1H-indole

MP:

### EXAMPLE 20

### (1H-Indol-2-yl)-(4-pyridin-2-yl-piperazin-1-yl)-methanone

MP: 198-200° C.

### EXAMPLE 21

### 2-(4-Pyridin-2-yl-piperazin-1-ylmethyl)-1H-indole

¹³C NMR (CDCl₃, 75 MHz) d 45.29, 53.03, 55.96, 77.44, 101.94, 107.29, 110.91, 113.52, 119.70, 120.28, 121.69, 128.40, 135.53, 136.37, 137.61, 148.00, 159.55.

¹H NMR (CDCl₃, 250 MHz) d 2.6 (m, 4H), 3.6 (m, 4H), 3.7 (s, 2H), 6.4 (s, 1H), 6.7 (m, 2H), 7.1-7.6 (m, 4H), 8.2 (m, 1H), 8.7 (br. s, 1H).

GC-MS, t_{R}^{=4.468} min., M=292, (M-162)=130.

### EXAMPLE 22

### (2′α,3′aβ,6′aβ)-1-(4-Fluoro-phenyl)-4-(5′-phenyl-1′,2′,3′,3′a,4′,6′a-hexahydro-pentalen-2′-yl)-piperazine dihydrochloride

MP: 250-253° C. Analysis calculated for C₂₄H₂₇FN₂.2 HCl.0.75H₂O: C, 66.28; H, 7.07; N, 6.44. Found: C, 66.18; H, 6.76; N, 6.56.

### EXAMPLE 23

### (2′α,3′aβ,5′α,6′aβ)-5′-[4-(4-Fluoro-phenyl)-piperazin-1-yl]-2′-phenyl-octahydro-pentalen-2′-ol maleate

MP: 206-207.5° C. Analysis calculated for C₂₄H₂₉FN₂O.0.75C₄H₄O₄.0.75H₂O: C, 67.41; H, 7.02; N, 5.82. Found: C, 67.24; H, 6.77; N, 5.68.

### EXAMPLE 24

### (2′α,3′aβ,5′α,6′aβ)-1-(4-Fluoro-phenyl)-4-(5′-phenyl-octahydro-pentalen-2′-yl)-piperazine dihydrochloride

MP: 255-256.5° C. Analysis calculated for C₂₄H₂₉FN₂.2HCl.0.25H₂O: C, 65.23; H, 7.18; N, 6.34. Found: C, 65.40; H, 7.02; N, 6.38.

### EXAMPLE 25

### (2′α,3′aβ,5′α,6′aβ)-2-Fluoro-4-[4-(5′-hydroxy-5′-phenyl-octahydro-pentalen-2′-yl)-piperazin-1-yl]-benzonitrile maleate

MP: 207-207.5° C. Analysis calculated for C₂₅H₂₈FN₃O.C₄H₄O₄: C, 66.78; H, 6.18; N, 8.06. Found: C, 66.64; H, 6.06; N, 8.14.

### EXAMPLE 26

### (2′α,3′aβ,5′α,6′aβ)-2-Fluoro-4-[4-(3′,3′a,4′,5′,6′,6′a-hexahydrospiro[isobenzofuran-1(3H),2′(1′H)-pentalen]-5′-yl)-1-piperazinyl]-benzonitrile maleate

MP: 221-221.5° C. Analysis calculated for C₂₆H₂₈FN₃O.C₄H₄O₄.0.5H₂O: C, 66.41; H, 6.13; N, 7.74. Found: C, 66.33; H, 6.26; N, 7.61.

### EXAMPLE 27

### (2′α,3′aβ,5′α,6′aβ)-5′-[4-(2-Methoxy-phenyl)-piperazin-1-yl]-2′-phenyl-octahydro-pentalen-2′-ol maleate

MP: 188-189° C. Analysis calculated for C₂₅H₃₂N₂O₂.C₄H₄O₄: C, 68.48; H, 7.13; N, 5.51. Found: C, 68.64; H, 7.10; N, 5.81.

### EXAMPLE 28

### (2′α,3′aβ,5′α,6′aβ)-2-(4-Fluoro-phenyl)-5′-[4-(5-fluoro-pyrimidin-2-yl)-piperazin-1-yl]-octahydro-pentalen-2′-ol maleate

MP: 219.5-220° C. Analysis calculated for C₂₂H₂₆F₂N₄O.C₄H₄O₄.0.5H₂O: C, 59.41; H, 5.94; N, 10.66. Found: C, 59.76; H, 5.89; N, 10.65.

### EXAMPLE 29

### (2′α,3′aβ,5′α,6′aβ)-2-Fluoro-4-{4-[5′-(4-fluoro-phenyl)-5′-hydroxy-octahydro-pentalen-2′-yl]-piperazin-1-yl}-benzonitrile maleate

MP: 204-204.5° C. Analysis calculated for C₂₅H₂₇F₂N₃O.C₄H₄O₄.H₂O: C, 62.47; H, 5.97; N, 7.54. Found: C, 62.77; H, 5.74; N, 7.58.

### EXAMPLE 30

### (2′α,3′aβ,5′α,6′aβ)-2′-(4-Fluoro-phenyl)-5′-[4-(4-fluoro-phenyl)-piperazin-1-yl]-octahydro-pentalen-2′-ol maleate

MP: 209-209.5° C. Analysis calculated for C₂₄H₂₈F₂N₂O.C₄H₄O₄: C, 65.36; H, 6.27; N, 5.54. Found: C, 65.65; H, 6.25; N, 5.34.

### EXAMPLE 31

### (2′α,3′aβ,6′aβ)-5-Fluoro-2-[4-(5′-phenyl-1′,2′,3′,3′a,4′,6′a-hexahydro-pentalen-2′-yl)-piperazin-1-yl]-pyrimidine maleate

MP: 202-203° C. Analysis calculated for C₂₂H₂₅FN₄.C₄H₄O₄: C, 64.99; H, 6.08; N, 11.66. Found: C, 64.67; H, 6.00; N, 11.79.

### EXAMPLE 32

### (2′α,3′aβ,6′aβ)-2-Fluoro-4-[4-(5′-phenyl-1′,2′,3′,3′a,4′,6′a-hexahydro-pentalen-2′-yl)-piperazin-1-yl]-benzonitrile maleate

MP: 172-173° C. Analysis calculated for C₂₅H₂₆FN₃.C₄H₄O₄: C, 69.17; H, 6.00; N, 8.34. Found: C, 69.06; H, 5.88; N, 8.57.

### EXAMPLE 33

### (2′α,3′aβ,5′α,6′aβ)-5-Fluoro-2-[4-(5′-phenyl-octahydro-pentalen-2′-yl)-piperazin-1-yl]-pyrimidine maleate

MP: 211.5-212° C. Analysis calculated for C₂₂H₂₇FN₄.C₄H₄O₄: C, 64.72; H, 6.48; N, 11.61. Found: C, 64.67; H, 6.43; N, 11.82.

### EXAMPLE 34

### (2′α,3′aβ,5′α,6′aβ)-2-Fluoro-4-[4-(5′-phenyl-octahydro-pentalen-2′-yl)-piperazin-1-yl]-benzonitrile maleate

MP: 195-196° C. Analysis calculated for C₂₅H₂₈FN₃.C₄H₄O₄: C, 68.89; H, 6.38; N, 8.31. Found: C, 68.99; H, 6.47; N, 8.30.

### EXAMPLE 35

### (2′α,3′aβ,5′α,6′aβ)-2-Fluoro-4-{4-[5′-(2-trifluoromethyl-phenyl)-octahydro-pentalen-2′-yl]-piperazin-1-yl}-benzonitrile maleate

MP: 192-193° C. Analysis calculated for C₂₆H₂₇F₄N₃.C₄H₄O₄: C, 62.82; H, 5.45; N, 7.33. Found: C, 62.87; H, 5.22; N, 7.27.

### EXAMPLE 36

### (2′α,3′aβ,6′aβ)-2-Fluoro-4-{4-[5-(2-methoxy-phenyl)-1′,2′,3′,3′a,4′,6′a-hexahydro-pentalen-2′-yl]-piperazin-1-yl}-benzonitrile maleate

MP: 155-156° C. Analysis calculated for C₂₆H₂₈FN₃O.C₄H₄O₄.0.25H₂O: C, 66.96; H, 6.09; N, 7.81. Found: C, 67.00; H, 6.05; N, 7.82.

### EXAMPLE 37

### (2′α,3′aβ,5′α,6′aβ)-2-Fluoro-4(4-[5′-(2-methoxy-phenyl)-octahydro-pentalen-2′-yl]-piperazin-1-yl]-benzonitrile maleate

MP: 176-177° C. Analysis calculated for C₂₆H₃₀FN₃O.C₄H₄O₄.0.50H₂O: C, 66.16; H, 6.48; N, 7.71. Found: C, 66.20; H, 6.31; N, 7.69.

### EXAMPLE 38

### (2′α,3′aβ,5′α,6′aβ)-2-Fluoro-4-{4-[5′-(1H-indol-3-yl)-octahydro-pentalen-2′-yl]-piperazin-1-yl}-benzonitrile maleate

MP: 226-227° C. Analysis calculated for C₂₇H₂₉FN₄.C₄H₄O₄: C, 68.37; H, 6.11; N, 10.29. Found: C, 68.17; H, 6.24; N, 10.20.

### EXAMPLE 39

### (2′α,3′aβ,5′α,6′aβ)-2-Fluoro-4-{4-[5′-(2-methanesulfonyl-phenyl)-octahydro-pentalen-2′-yl]-piperazin-1-yl}-benzonitrile maleate

MP: 179-180° C. Analysis calculated for C₂₆H₃₀FN₃O₂S.C₄H₄O₄.0.25H₂O: C, 61.25; H, 5.91; N, 7.14. Found: C, 61.26; H, 6.32; N, 6.76.

### EXAMPLE 40

### (2′α,3′aβ,5′β,6′aβ)-2-Fluoro-4-[4-(3′,3′a,4′,5′,6′,6′a-hexahydrospiro[isobenzofuran-1(3H),2′(1′H)-pentalen]-5′-yl)-1-piperazinyl]-benzonitrile maleate

MP >260° C. Analysis calculated for C₂₆H₂₈FN₃O.CH₄O₃S: C, 63.14; H, 6.27; N, 8.18. Found: C, 63.12; H, 6.66; N, 8.00.

### EXAMPLE 41

### (2′α,3′aβ,5′α,6′aβ)-2-Fluoro-4-[4-(3,3′,3′a,4,4′,5′,6′,6′a-hexahydrospiro[2H-1-benzopyran-2,2′(1′H)-pentalen]-5′-yl)-1-piperazinyl]-benzonitrile maleate

MP: 176-177° C. Analysis calculated for C₂₇H₂₈FN₃O₂.C₄H₄O₄.0.50H₂O: C, 65.25; H, 5.82; N, 7.36. Found: C, 65.52; H, 6.06; N, 7.19.

### EXAMPLE 42

### (2′α,3′aβ,5′β,6′aβ)-2-Fluoro-4-[4-(3,3′,3′a,4,4′,5′,6′,6′a-hexahydrospiro[2H-1-benzopyran-2,2′(1′H)-pentalen]-5′-yl)-1-piperazinyl]-benzonitrile maleate

MP: 179-180° C. Analysis calculated for C₂₇H₂₈FN₃O₂.C₄H₄O₄: C, 66.30; H, 5.74; N, 7.48. Found: C, 66.17; H, 6.07; N, 7.34.

### EXAMPLE 43

### (2′α,3′aβ,5′α,6′aβ)-2-Fluoro-4-{4-[5′-(2-trifluoromethoxy-phenyl)-octahydro-pentalen-2′-yl]-piperazin-1-yl}-benzonitrile maleate

MP: 126-129° C. NMR DMSO d₆ δ 7.70 (t, J=8.5 Hz, 1H), 7.52 (d, J=7.1 Hz, 1H), 7.40-7.25 (m, 3H), 7.09 (d, J=13.6 Hz, 1H), 6.96 (d, J=9.0 Hz, 1H), 6.06 (s, 2H), 3.73-2.90 (br m, 10H), 2.65-2.54 (m, partially under DMSO, 1H), 2.46-2.18 (m, 4H), 1.63-1.42 (m, 4H).

### EXAMPLE 44

### (2′α,3′aβ,5′α,6′aβ)-2-Fluoro-4-{4-[5′-(2-fluoro-phenyl)-octahydro-pentalen-2′-yl]-piperazin-1-yl}-benzonitrile maleate

MP: 179-180.5° C. Analysis calculated for C₂₅H₂₇F₂N₃.C₄H₄O₄: C, 66.53; H, 5.97; N, 8.03. Found: C, 66.62; H, 6.24; N, 7.98.

### EXAMPLE 45

### (2′α,3′aβ,5′α,6′aβ)-2-Cyano-4-{4-[5′-(2-fluoro-phenyl)-octahydro-pentalen-2′-yl]-piperazin-1-yl}-benzonitrile maleate

MP: 193-194° C. Analysis calculated for C₂₆H₂₇FN₄.C₄H₄O₄.0.50H₂O: C, 66.78; H, 5.98; N, 10.38. Found: C, 66.99; H, 6.05; N, 10.34.

### EXAMPLE 46

### (2′α,3′aβ,5′α,6′aβ)-2-Fluoro-4-[4-(5′-pyridin-2-yl-octahydro-pentalen-2′-yl)-piperazin-1-yl]-benzonitrile dihydrochloride

MP: 203-206° C. Analysis calculated for C₂₄H₂₇FN₄.2HCl.H₂O: C, 59.88; H, 6.49; N, 11.63. Found: C, 59.55; H, 6.42; N, 11.47.

### EXAMPLE 47

### (2′α,3′aβ,5′α,6′aβ)-5-Fluoro-2-{4-[5′-(2-methoxy-phenyl)-octahydro-pentalen-2′-yl]-piperazin-1-yl}-pyrimidine maleate

MP: 183.5-184.5° C. Analysis calculated for C₂₃H₂₉FN₄O.C₄H₄O₄: C, 63.26; H, 6.49; N, 10.93. Found: C, 63.21; H, 6.71; N, 10.82.

### EXAMPLE 48

### (2′α,3′aβ,5′α,6′aβ)-2-Fluoro-4-{4-[5′-(6-fluoro-2-oxo-2,3-dihydro-benzoimidazol-1-yl)-octahydro-pentalen-2′-yl]-piperazin-1-yl}-benzonitrile dimesylate

MP: 219-222° C. Analysis calculated for C₂₆H₂₇FN₅O.2CH₄O₃S: C, 51.29; H, 5.38; N, 10.68. Found: C, 51.84; H, 5.57; N, 10.64.

### EXAMPLE 49

### (2′α,3′aβ,5′α,6′aβ)-2-Fluoro-4-{4-[5′-(6-fluoro-2-methylbenzoimidazol-1-yl)-octahydro-pentalen-2′-yl]-piperazin-1-yl}-benzonitrile dimesylate

MP: >260° C. Analysis calculated for C₂₇H₂₉F₂N₅.2CH₄O₃S.0.50H₂O: C, 52.56; H, 5.48; N, 10.57. Found: C, 52.64; H, 5.71; N, 10.57.

### EXAMPLE 50

### (2′α,3′aβ,5′α,6′aβ)-5-Fluoro-2-[4-(3′,3′a,4′,5′,6′,6′a-hexahydrospiro[isobenzofuran-1(3H),2′(1′H)-pentalen]-5′-yl)-piperazin-1-yl]-pyrimidine

MP=186° C. NMR CDCl₃ δ 8.20 (s, 2H), 7.25-7.17 (m, 4H), 7.12-7.09 (m, 1H), 5.00 (s, 2H), 3.79-3.71 (m, 4H), 2.72-2.44 (m, 7H), 2.20-2.13 (m, 2H), 2.17-1.93 (m, 2H), 1.69-1.67 (s, 2H).

### EXAMPLE 51

### (2′β,3′aβ,5′α,6′aβ)-5-Fluoro-2-[4-(3′,3′a,4′,5′,6′,6′a-hexahydrospiro[isobenzofuran-1(3H),2′(1′H)-pentalen]-5′-yl)-piperazin-1-yl]-pyrimidine

MP: 186-187° C. NMR CDCl₃ δ 8.18 (s, 2H), 7.26-7.10 (m, 3H), 7.08-7.06 (m, 1H), 5.00 (s, 2H), 3.78-3.76 (br s, 4H), 2.78-2.73 (m, 2H), 2.66-2.54 (m, 5H), 2.32-2.22 (m, 4H), 1.74-1.69 (m, 2H), 1.38-1.29 (m, 2H).

### EXAMPLE 52

### (2′α,3′aβ,5′α,6′aβ)-1-Phenyl-4-(3,3′,3′a,4,4′,5′,6′,6′a-hexahydrospiro[2H-1-benzopyran-2,2′(1′H)-pentalen]-5′-yl]-5′-yl)-piperazine maleate

MP: 200-201° C. Analysis calculated for C₂₆H₃₀N₂O₂.C₄H₄O₄: C, 69.48; H, 6.61; N, 5.40. Found: C, 69.48; H, 6.80; N, 5.44.

### EXAMPLE 53

### (2′β,3′aβ,5′α,6′aβ)-1-Phenyl-4-(3,3′,3′a,4,4′,5′,6′,6′a-hexahydrospiro[2H-1-benzopyran-2,2′(1′H)-pentalen]-5′-yl]-5′-yl)-piperazine maleate

MP: 220-221° C. Analysis calculated for C₂₆H₃₀N₂O₂.C₄H₄O₄: C, 69.48; H, 6.61; N, 5.40. Found: C, 69.28; H, 6.84; N, 5.33.

### EXAMPLE 54

### (2′α,3′aβ,5′α,6′aβ)-3-[5′-(4-Phenyl-piperazin-1-yl)-octahydro-pentalen-2′-yl]-1H-indole maleate

MP: 232-232.5° C. Analysis calculated for C₂₆H₃₁N₃.C₄H₄O₄: C, 71.83; H, 7.03; N, 8.38. Found: C, 71.57; H, 7.38; N, 8.31.

### EXAMPLE 55

### (2′α,3′aβ,6′aβ)-1-Phenyl-4-(5′-phenyl-1′,2′,3′,3′a,4′,6′a-hexahydro-pentalen-2′-yl)-piperazine dimaleate

MP: 156-157° C. Analysis calculated for C₂₆H₃₀N₂O₂.2C₄H₄O₄: C, 66.65; H, 6.29; N, 4.86. Found: C, 66.27; H, 6.57; N, 5.00.

### EXAMPLE 56

### (2′α,3′aβ,5′α,6′aβ)-1-Phenyl-4-(5′-phenyl-octahydro-pentalen-2′-yl)-piperazine maleate

MP: 217-218° C. Analysis calculated for C₂₄H₃₀N₂.C₄H₄O₄: C, 72.70; H, 7.41; N, 6.06. Found: C, 72.28; H, 7.46; N, 6.01.

### EXAMPLE 57

### (2′α,3′aβ,5′α,6′aβ)-6-Fluoro-2-methyl-1-[5′-(4-phenyl-piperazin-1-yl)-octahydro-pentalen-2′-yl]-1H-benzoimidazole dimaleate

MP: 203-205° C. Analysis calculated for C₂₆H₃₁FN₄.2C₄H₄O₄.0.50H₂O: C, 61.90; H, 6.11; N, 8.49. Found: C, 61.96; H, 6.01; N, 8.58.

### EXAMPLE 58

### (2′α,3′aβ,5′β,6′aβ)-1-[5′-(4-Fluoro-phenoxy)-octahydro-pentalen-2′-yl]-4-phenyl-piperazine maleate

MP: 177-178° C. Analysis calculated for C₂₄H₂₉FN₂O.C₄H₄O₄: C, 67.72; H, 6.70; N, 5.64. Found: C, 67.33; H, 6.82; N, 5.62.

### EXAMPLE 59

### (2′α,3′aβ,5′β,6′aβ)-2-[5′-(4-Phenyl-piperazin-1-yl)-octahydro-pentalen-2′-yl]-isoindole-1,3-dione maleate

MP: 235.5-236° C. Analysis calculated for C₂₆H₂₉N₃O₂.C₄H₄O₄: C, 67.78; H, 6.26; N, 7.90. Found: C, 67.71; H, 6.37; N, 7.94.

### EXAMPLE 60

### (2′α,3′aβ,5′α,6′aβ)-N-(2-{5′-[4-(5-Fluoro-pyrimidin-2-yl)-piperazin-1-yl]-octahydro-pentalen-2′-yl}-phenyl)-acetamide maleate

MP: 211.5-212° C. Analysis calculated for C₂₄H₃₀FN₅O.C₄H₄O₄: C, 62.33; H, 6.35; N, 12.98. Found: C, 62.07; H, 6.32; N, 12.87.

### EXAMPLE 61

### (2′α,3′aβ,′5α,6′aβ)-N-(2-{5′-[4-(4-Cyano-3-fluoro-phenyl)-piperazin-1yl]-octahydro-pentalen-2′-yl}-phenyl)-acetamide maleate

MP: 197-199° C. Analysis calculated for C₂₇H₃₁FN₄O.C₄H₄O₄: C, 66.18; H, 6.27; N, 9.96. Found: C, 66.06; H, 6.20; N, 9.89.

### EXAMPLE 62

### (2′α,3′aβ,5′α,6′aβ)-2-Fluoro-4-{4-[5′-(2-oxo-2,3-dihydro-benzoimidazol-1-yl)-octahydro-pentalen-2′-yl]-piperazin-1-yl}-benzonitrile mesylate

MP >260° C. Analysis calculated for C₂₆H₂₈FN₅O.CH₄O₃S.0.50H₂O: C, 58.89; H, 6.04; N, 12.72. Found: C, 59.01; H, 6.06; N, 12.71.

### EXAMPLE 63

### (2′α,3′aβ,5′α,6′aβ)-1-{5′-[4-(5-Fluoro-pyrimidin-2-yl)-piperazin-1-yl]-octahydro-pentalen-2′-yl}-1,3-dihydro-benzoimidazol-2-one mesylate

MP >260° C. Analysis calculated for C₂₃H₂₇FN₆O.CH₄O₃S: C, 55.58; H, 6.04; N, 16.20. Found: C, 55.48; H, 5.87; N, 16.41.

### EXAMPLE 64

### (2′α,3′aβ,5′α,6′aβ)-2-{5′-[4-(4-Cyano-3-fluoro-phenyl)-piperazin-1-yl]-octahydro-pentalen-2′-yl}-benzamide maleate

MP 198.5-200° C. Analysis calculated for C₂₆H₂₉FN₄O.C₄H₄O₄.0.50H₂O: C, 64.62; H, 6.15; N, 10.05. Found: C, 64.84; H, 6.01; N, 10.03.

### EXAMPLE 65

### (2′α,3′aβ,5′α,6′aβ)-N-[5′-(4-Phenyl-piperazin-1-yl)-octahydro-pentalen-2′-yl]-benzamide maleate

MP: 211-212.5° C. Analysis calculated for C₂₅H₃₁N₃O.C₄H₄O₄.0.25H₂O: C, 68.28; H, 7.01; N, 8.23. Found: C, 68.17; H, 6.94; N, 8.18.

### EXAMPLE 66

### (2′α,3′aβ,5′β,6′aβ)-2-Fluoro-44-[5′-(4-fluoro-phenoxy)-octahydro-pentalen-2′-yl]-piperazin-1-yl)-benzonitrile maleate

MP: 192-193° C. Analysis calculated for C₂₅H₂₇F₂N₃O.C₄H₄O₄: C, 64.55; H, 5.79; N, 7.79. Found: C, 64.50; H, 5.80; N, 7.71.

### EXAMPLE 67

### (2′α,3′aβ,5′β,6′aβ)-5-Fluoro-2-(4-[5′-(4-fluoro-phenoxy)-octahydro-pentalen-2′-yl]-piperazin-1-yl)-pyrimidine maleate

MP: 192-194° C. Analysis calculated for C₂₂H₂₆F₂N₄O.C₄H₄O₄: C, 60.46; H, 5.85; N, 10.85. Found: C, 60.30; H, 5.82; N, 10.78.

### EXAMPLE 68

### (2′α,3′aβ,5′β,6′aβ)-2-Fluoro-4-{4-[5′-(2-oxo-2,3-dihydro-benzoimidazol-1-yl)-octahydro-pentalen-2′-yl]-piperazin-1-yl}-benzonitrile maleate

MP: 170-177° C. NMR DMSO d₆ δ 10.89 (s, 1H), 7.70 (t, J=8.4 Hz, 1H), 7.30-7.23 (m, 1H), 7.11 (d, J=13.9 Hz, 1H), 7.04-6.94 (m, 4H), 6.06 (s, 2H), 4.97-4.82 (m, 1H), 3.62-2.80 (br m, 10H), 2.75-2.63 (m, 2H), 2.60-2.50 (m partially under DMSO peak, 1H), 2.48-2.36 (m, 2H), 1.60 (dd, J₁=12.4 Hz, J₂=6.6 Hz, 2H), 1.58-1.34 (m, 2H).

### EXAMPLE 69

### (2′α,3′aβ,5′α,6′aβ)-2-Fluoro-4-{4-[5′-(3-methoxy-phenyl)-octahydro-pentalen-2′-yl]-piperazin-1-yl}-benzonitrile maleate

MP: 169-170° C. Analysis calculated for C₂₆H₃₀FN₃O.C₄H₄O₄: C, 67.27; H, 6.40; N, 7.85. Found: C, 67.18; H, 6.52; N, 7.87.

### EXAMPLE 70

### (2′α,3′aβ,5′α,6′aβ)-2-Fluoro-4-{4-[5′-(4-methoxy-phenyl)-octahydro-pentalen-2′-yl]-piperazin-1-yl}-benzonitrile maleate

MP: 186-186.5° C. Analysis calculated for C₂₆H₃₀FN₃O.C₄H₄O₄.0.25H₂O: C, 66.71; H, 6.44; N, 7.78. Found: C, 66.70; H, 6.60; N, 7.60.

### EXAMPLE 71

### (2′α,3′aβ,5′α,6′aβ)-2-Fluoro-4-[4-(5′-m-tolyl-octahydro-pentalen-2′-yl)-piperazin-1-yl]-benzonitrile maleate

MP: 198-198.5° C. Analysis calculated for C₂₆H₃₀FN₃.C₄H₄O₄: C, 69.35; H, 6.60; N, 8.09. Found: C, 69.48; H, 6.74; N, 8.14.

### EXAMPLE 72

### (2′α,3′aβ,5′α,6′aβ)-2-Fluoro-4-[4-(5′-p-tolyl-octahydro-pentalen-2′-yl)-piperazin-1-yl]-benzonitrile maleate

MP: 194-195° C. NMR DMSO d₆ δ 7.70 (t, J=8.5 Hz, 1H), 7.16-7.09 (m, 5H), 6.96)d, J=8.7 Hz, 1H), 6.06 (s, 2H), 3.75-2.85 (m, 11H), 2.55-2.43 (m partially under DMSO peak, 1H), 2.40-2.23 (m with singlet @ 2.26, 7H total), 1.63-1.32 (m, 4H).

### EXAMPLE 73

### (2′β,3′aβ,5′β,6′aβ)-1-[5′-(4-Fluoro-phenoxy)-octahydro-pentalen-2′-yl]-4-phenyl-piperazine maleate

MP: 174-175° C. Analysis calculated for C₂₄H₂₉FN₂O.C₄H₄O₄: C, 67.72; H, 6.70; N, 5.64. Found: C, 67.82; H, 6.83; N, 5.59.

### EXAMPLE 74

### (2′α,3′aβ,5′α,6′aβ)-2-Fluoro-4-[4-(5′-o-tolyl-octahydro-pentalen-2′-yl)-piperazin-1-yl]-benzonitrile maleate

MP: 198-199° C. Analysis calculated for C₂₆H₃₀FN₃.C₄H₄O₄: C, 69.35; H, 6.60; N, 8.09. Found: C, 69.13; H, 6.69; N, 8.12.

### EXAMPLE 75

### (2′α,3′aβ,5′α,6′aβ)-1-Phenyl-4-[5′-(3-pyrrolidin-1-ylmethyl-phenyl)-octahydro-pentalen-2′-yl]-piperazine dimaleate

MP: 163.5-164° C. Analysis calculated for C₂₉H₃₉N₃.2C₄H₄O₄: C, 67.15; H, 7.16; N, 6.35. Found: C, 66.81; H, 7.22; N, 6.27.

### EXAMPLE 76

### (2′α,3′aβ,5′α,6′aβ)-5-Fluoro-2-[4-(3′,3′a,4′,5′,6′,6′a-hexahydro-3′a,6′a-dimethylspiro[isobenzofuran-1(3H),2′(1′H)-pentalen]-5′-yl)-1-piperazinyl]-pyrimidine maleate

MP: 224.5-225° C. Analysis calculated for C₂₅H₃₁FN₄O.C₄H₄O₄.0.25H₂O: C, 64.13; H, 6.59; N, 10.32. Found: C, 64.25; H, 6.68; N, 10.14.

### EXAMPLE 77

### (2′β,3′aβ,5′α,6′aβ)-5-Fluoro-2-[4-(3′,3′a,4′,5′,6′,6′a-hexahydro-3′a,6′a-dimethylspiro[isobenzofuran-1(3H),2′(1′H)-pentalen]-5′-yl)-1-piperazinyl]-pyrimidine maleate

MP: 222-223° C. NMR DMSO d₆ δ 8.58 (s, 2H), 7.34-7.30 (m, 1H), 7.28-7.25 (m, 3H), 6.04 (s, 2H), 4.94 (s, 2H), 3.65-2.75 (br m, 9H), 2.20-2.12 (m, 2H), 1.94 (AB quartet, Δᵥ=37.8 Hz, J=13.2 Hz, 4H), 1.54 (br t, J=11.7 Hz, 2H), 1.21 (s, 6H).

### EXAMPLE 78

### (2′α,3′aβ,5′β,6′aβ)-4-{4-[5′-(1,3-Dioxo-1,3-dihydro-isoindol-2-yl)-octahydro-pentalen-2′-yl]-piperazin-1-yl}-2-fluoro-benzonitrile maleate

MP: 224-224.5° C. Analysis calculated for C₂₇H₂₇FN₄O₂.C₄H₄O₄: C, 64.80; H, 5.44; N, 9.75. Found: C, 64.85; H, 5.56; N, 9.74.

### EXAMPLE 79

### (2′α,3′aβ,5′β,6′aβ)-2-{5′-[4-(5-Fluoro-pyrimidin-2-yl)-piperazin-1-yl]-octahydro-pentalen-2′-yl}-isoindole-1,3-dione maleate

MP: 241.5-242° C. Analysis calculated for C₂₄H₂₆FN₅O₂.C₄H₄O₄: C, 60.97; H, 5.48; N, 12.70. Found: C, 60.66; H, 5.55; N, 12.44.

### EXAMPLE 80

### (2′α,3′aβ,5′α,6′aβ)-2-Fluoro-4-[4-(3,3′,3′a,4,4′,5′,6′,6′a-hexahydrospiro[2H-6-fluoro-1-benzopyran-2,2′(1′H)-pentalen]-5′-yl]-5′-yl)-1-piperazinyl]-benzonitrile maleate

MP: 219-220° C. Analysis calculated for C₂₄H₂₆F₂N₄O₂.C₄H₄O₄.0.50H₂O: C, 59.46; H, 5.55; N, 9.90. Found: C, 59.86; H, 5.70; N, 9.40.

### EXAMPLE 81

### (2′β,3′aβ,5′α,6′aβ)-2-Fluoro-4-[4-(3,3′,3′a,4,4′,5′,6′,6′a-hexahydrospiro[2H-6-fluoro-1-benzopyran-2,2′(1′H)-pentalen]-5′-yl]-5′-yl)-1-piperazinyl]-benzonitrile maleate

MP: 216.5-217° C. Analysis calculated for C₂₄H₂₆F₂N₄O₂.C₄H₄O₄: C, 60.43; H, 5.43; N, 10.07. Found: C, 60.39; H, 5.47; N, 9.90.

### EXAMPLE 82

### (2′α,3′aβ,5′α,6′aβ)-5-Fluoro-2-[4-(5′-o-tolyl-octahydro-pentalen-2′-yl)-piperazin-1-yl]-pyrimidine maleate

MP: 204-205° C. Analysis calculated for C₂₃H₂₉FN₄.C₄H₄O₄: C, 65.31; H, 6.70; N, 11.28. Found: C, 65.38; H, 6.77; N, 11.32.

### EXAMPLE 83

### (2′β,3′aβ,5′α,6′aβ)-1-{5′-[4-(4-Fluoro-phenyl)-piperazin-1-yl]-octahydro-pentalen-2′-yl}-1,3-dihydro-benzoimidazol-2-one maleate

MP: 217-218° C. Analysis calculated for C₂₅H₂₉FN₄O.C₄H₄O₄: C, 64.91; H, 6.20; N, 10.44. Found: C, 64.57; H, 6.28; N, 10.18.

### EXAMPLE 84

### (2′β,3′aβ,5′α,6′aβ)-2-[5′-(4-Phenyl-piperazin-1-yl)-octahydro-pentalen-2′-yloxy]-1H-benzoimidazole maleate

MP: 161-162° C. Analysis calculated for C₂₅H₃₀N₄O.C₄H₄O₄: C, 67.16; H, 6.61; N, 10.80. Found: C, 67.05; H, 6.66; N, 10.59.

### EXAMPLE 85

### (2′α,3′aβ,5′α,6′aβ0-5-Chloro-2-{4-[5′-(2-methoxy-phenyl)-octahydro-pentalen-2′-yl]-piperazin-1-yl}-pyrimidine maleate

MP: 199.5-200° C. Analysis calculated for C₂₃H₂₉ClN₄O.C₄H₄O₄: C, 61.30; H, 6.29; N, 10.59. Found: C, 61.05; H, 6.31; N, 10.83.

### EXAMPLE 86

### (2′α,3′aβ,5′α,6′aβ)-5-Chloro-2-[4-(5′-o-tolyl-octahydro-pentalen-2′-yl)-piperazin-1-yl]-pyrimidine maleate

MP: 200-200.5° C. Analysis calculated for C₂₃H₂₉ClN₄.C₄H₄O₄: C, 63.21; H, 6.48; N, 10.92. Found: C, 62.97; H, 6.33; N, 11.29.

### EXAMPLE 87

### (2′β,3′aβ,5′α,6′aβ)-2-{5′-[4-(3,4-Difluoro-phenyl)-piperazin-1-yl]-octahydro-pentalen-2′-yl}-isoindole-1,3-dione maleate

MP: 221.5-222° C. Analysis calculated for C₂₆H₂₇F₂N₃O₂.C₄H₄O₄: C, 63.48; H, 5.51; N, 7.46. Found: C, 63.28; H, 5.51; N, 7.64.

### EXAMPLE 88

### (2′β,3′aβ,5′α,6′aβ)-2-{5′-[4-(4-Fluoro-phenyl)-piperazin-1-yl]-octahydro-pentalen-2′-yl}-isoindole-1,3-dione maleate

MP: 209-210?C. Analysis calculated for C₂₆H₂₈FN₃O₂.C₄H₄O₄.0.50H₂O: C, 64.51; H, 5.95; N, 7.52. Found: C, 64.47; H, 5.91; N, 7.66.

### EXAMPLE 89

### (2′β,3′aβ,5′α,6′aβ)-1-{5′-[4-(3,4-Difluoro-phenyl)-piperazin-1-yl]-octahydro-pentalen-2′-yl}-1,3-dihydro-benzoimidazol-2-one maleate

MP: 201-202° C. Analysis calculated for C₂₅H₂₈F₂N₄O.C₄H₄O₄.0.50H₂O: C, 61.80; H, 5.90; N, 9.94. Found: C, 62.10; H, 5.80; N, 9.56.

1. A compound of the formula 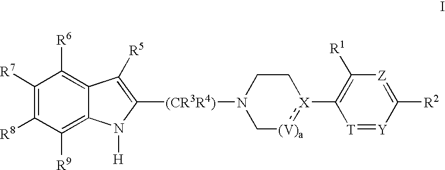
or the pharmaceutically acceptable salt thereof, wherein the broken line represents an optional double bond;
a is 0 or 1, wherein when a is 0, X may form an optional double bond with the carbon adjacent to V;
V is CHR¹⁰ wherein R¹⁰ is hydrogen or (C₁-C₆)alkyl;
T is nitrogen or CH;
X is nitrogen or CR¹¹ wherein R¹¹ is hydrogen, (C₁-C₆)alkyl, (C₁-C₆)alkoxy, hydroxy or cyano;
Y and Z are each independently nitrogen or CR¹² wherein R¹² is hydrogen, chloro, bromo, trifluoromethyl, trifluoromethoxy, cyano, (C₁-C₆)alkoxy or (C₁-C₆)alkyl;
R¹ is hydrogen, fluoro, chloro, bromo, trifluoromethyl, trifluoromethoxy, cyano or (C₁-C₆)alkyl;
R², R⁶, R⁷, R³ and R⁹ are each independently selected from hydrogen, fluoro, chloro, bromo, trifluoromethyl, trifluoromethoxy, cyano, (C₁-C₆)alkoxy and. (C₁-C₆)alkyl;
R³ and R⁴ are each independently hydrogen or (C₁-C₆)alkyl; and
R⁵ is hydrogen, (C₁-C₆)alkoxy, trifluoromethyl, cyano, (C₁-C₆)alkyl or R¹³CO— wherein R³ is amino, (C₁-C₆)alkylamino, ((C₁-C₆)alkyl)₂amino, (C₁-C₆)alkyl, (C₆-C₁₀)aryl;
or when a is 1, R¹ and R¹⁰ may be taken together with the carbons to which they are attached to form a compound of the formula 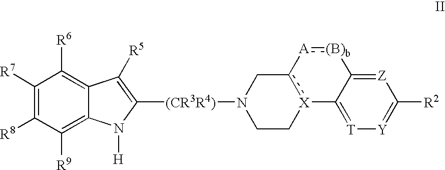
wherein the broken lines represent optional bonds;
T, X, Y, Z, R², R³, R⁴, R⁵, R⁶, R⁷, R³ and R⁹ are defined as above;
b is 0 or 1; and
A and B are each independently CH, CH₂, oxygen, sulfur, NH or nitrogen;
with the proviso that when X is nitrogen, the optional double bond between X and V does not exist;
with the proviso that when b is 0, the optional double bond between A and B does not exist; and
with the proviso that when b is 1, A and B cannot both be oxygen or sulfur.

2. A compound according to claim 1, wherein X is nitrogen.

3. A compound according to claim 1, wherein Y and Z are each CR¹² wherein R¹² is hydrogen or fluoro.

4. A compound according to claim 1, wherein R² is hydrogen, fluoro or chloro.

5. A compound according to claim 1, wherein R³, R⁴ and R⁵ are hydrogen.

6. A compound according to claim 1, wherein R⁷ is fluoro or chloro.

7. A compound according to claim 1, wherein R⁹ is fluoro, chloro, bromo or alkoxy.

8. A compound according to claim 1, wherein X is nitrogen; Y and Z are each CR¹² wherein R¹² is hydrogen or fluoro; R² is hydrogen fluoro or chloro; R³, R⁴ and. R⁵ are hydrogen; R⁷ is fluoro or chloro; and R⁷ is fluoro, chloro, bromo or alkoxy.

9. A compound according to claim 1, wherein said compound is selected from the group consisting of:
2-[4-(3-Trifluoromethyl-phenyl)-piperazin-1-ylmethyl]-1H-indole;
5-Fluoro-2-[4-(3-trifluoromethyl-phenyl)-piperazin-1-ylmethyl]-1H-indole;
5-Fluoro-2-[4-(4-fluoro-phenyl)-piperazin-1-ylmethyl]-1H-indole;
5-Fluoro-2-[4-(4-fluoro-phenyl)-piperazin-1-ylmethyl]-1H-indole;
5-Fluoro-2-(4-pyridin-2-yl-piperazin-1-ylmethyl)-1H-indole;
2-[4-(6-Chloro-pyridazin-3-yl)-piperazin-1-ylmethyl]-5-fluoro-1H-indole;
5-Fluoro-2-(4-[5′-fluoro]pyridin-2-yl-piperazin-1-ylmethyl)-1H-indole;
2-(4-pyridin-2-yl-piperazin-1-ylmethyl)-1H-azaindole;
5-Fluoro-2-(4-pyridin-2-yl-piperazin-1-ylmethyl)-1H-azaindole; and
2-[4-(4-fluoro-phenyl)-piperazin-1-ylmethyl]-1H-azaindole.

10. A method for treating a disorder of the dopamine system in a mammal, comprising administering to said mammal an amount of a D4 dopamine receptor selective compound according to claim 1, or a pharmaceutically acceptable salt thereof, that is effective in treating such disorder.

11. A method according to claim 10, wherein disorders of the dopamine system include psychotic disorders, movement disorders, gastrointestinal disorders, chemical abuse, chemical dependencies, substance abuse, vascular and cardiovascular disorders, ocular disorders and sleep disorders.

12. A method for treating a disorder of the dopamine system in a mammal, comprising administering to said mammal an amount of a D4 dopamine receptor selective compound according to claim 1, or a pharmaceutically acceptable salt thereof, in conjunction with one or more D1, D2, D3 or D5 dopamine receptor agonists, that is effective in treating such disorder.

13. A method according to claim 12, wherein disorders of the dopamine system include psychotic disorders, movement disorders, gastrointestinal disorders, chemical abuse, chemical dependencies, substance abuse, vascular and cardiovascular disorders, ocular disordrs and sleep disorders.

14. A method according to claim 11, wherein psychotic disorders include affective psychosis, schizophrenia, and schizoaffective disorders.

15. A method according to claim 11, wherein movement disorders include extrapyramidal side effects from neuroleptic agents, neuroleptic malignant syndrome, tardive dyskinesia, Gilles De La Tourette's syndrome, Parkinson's disease or Huntington's disease.

16. A method according to claim 11, wherein gastrointestinal disorders include gastric acid secretion or emesis.

17. A method according to claim 11, wherein vascular and cardiovascular disorders include congestive heart failure and hypertension.

18. A pharmaceutical composition for treating a disorder of the dopamine system in a mammal, comprising administering to said mammal an amount of a D4 dopamine receptor selective compound according to claim 1, or a pharmaceutically acceptable salt thereof, that is effective in treating such disorder.

19. A pharmaceutical composition according to claim 18, wherein disorders of the dopamine system include psychotic disorders, movement disorders, gastrointestinal disorders, chemical abuse, chemical dependencies, substance abuse, vascular and cardiovascular disorders, ocular disorders and sleep disorders.

20. A pharmaceutical composition for treating a disorder of the dopamine system in a mammal, comprising administering to said mammal an amount of a D4 dopamine receptor selective compound according to claim 1, or a pharmaceutically acceptable salt thereof, in conjunction with one or more D1, D2, D3 or D5 dopamine receptor agonists, that is effective in treating such disorder.

21. A pharmaceutical composition according to claim 20, wherein disorders of the dopamine system include psychotic disorders, movement disorders, gastrointestinal disorders, chemical abuse, chemical dependencies, substance abuse, vascular and cardiovascular disorders, ocular disorders and sleep disorders.
